(19) United States
(12) Patent Application Publication
(10) Pub. No.: US 2010/0016749 A1
(43) Pub. Date: Jan. 21, 2010

(54) Method and System for the Monitoring of Respiratory Acitivity and for the Treatment of Breathing Disorders Such as Sleep Apnea

(75) Inventors: Willem Atsma, Quebec (CA); Nader Kameli, Hugo, MN (US)

Correspondence Address: POLSINELLI SHUGHART PC, 700 W. 47TH STREET, SUITE 1000, KANSAS CITY, MO, 64112-1802, US

(73) Assignee: VICTHOM HUMAN BIONICS, INC., Saint-Augustin-de-Desmaures, QC (CA)

(21) Appl. No.: 11/920,795

(22) PCT Filed: Sep. 19, 2007
(86) PCT No.: PCT/CA2007/001784

§ 371 Date: Jul. 3, 2008

Related U.S. Application Data
(60) Provisional application No. 60/845,515, filed on Sep. 19, 2006.

(51) Int. Cl. A61B 5/08

(52) U.S. Cl. 600/529

(57) ABSTRACT

A method and system for sensing the vagus nerve in order to monitor respiratory activity, treating breathing disorders, such as, for example, sleep apnea and a generic bio-interfacing platform that may be adapted for either open-loop or closed-loop applications.

### CROSS-REFERENCE TO RELATED APPLICATION

This application claims the benefits of U.S. provisional patent applications No. 60/845,515 filed Sep. 19, 2006; which is hereby incorporated by reference.

### TECHNICAL FIELD

The present invention relates to a method and system for the monitoring of respiratory activity. The present invention further relates to a method and system for the treatment of breathing disorders, more specifically to sleep apnea.

### BACKGROUND

The use of implantable devices for the monitoring of breathing activity as well as closed-loop implantable devices including sensing and stimulating systems directly interfaced with the peripheral nervous system for providing therapeutic electrical signals may provide advantageous effects to subjects who present breathing disorders that may be mitigated or circumvented by the use of “adaptive” stimulation signals whose characteristics are in direct relation with the sensing signal. One such breathing disorder is sleep apnea.

Sleep apnea is defined as an intermittent cessation of airflow in the airways during sleep. By convention, apneas of at least 10 seconds duration have been considered important, but in most subjects the apneas are 20 to 30 seconds in duration and may be as long as 2 to 3 minutes. There is uncertainty as to the minimum number of apneas that should be considered clinically important, although by the time most subjects come to attention they have at least 10 to 15 events per hour of sleep and may even have up to 400-600 events in an 8-hour period of sleep.

During an event oxygen saturation drops and the heart rate slows. A subject will not awaken from an apnea event, but his sleeping patterns change. The percentage of sleep time spent in stage-1 non-rapid eye movement (REM) sleep, which is normally 10% or less, can increase to 30-50%. At the end of an apnea event, a subject will partially wake up and enter a different stage of sleep. The brief arousals from sleep will reduce the restorative effect of sleep and result in excessive daytime sleepiness.

The clinical importance of sleep apnea arises from the fact that it is one of the leading causes of excessive daytime sleepiness. Indeed, epidemiologic studies have established a prevalence of clinically important sleep apnea of at least two percent in middle-aged women and four percent in middle-aged men.

Sleep apneas have been classified into three types: central, obstructive, and mixed. In central sleep apnea (CSA) the neural drive to all the respiratory muscles is transiently abolished. In contrast, in obstructive sleep apnea (OSA) airflow ceases despite continuing respiratory drive because of occlusion of the oropharyngeal airway. Mixed apneas, which consist of a central apnea followed by an obstructive component, are a variant of OSA.

The definitive event in CSA is transient abolition of central drive to the ventilatory muscles. The resulting apnea leads to a primary sequence of events similar to those of OSA. Several underlying mechanisms can result in cessation of respiratory drive during sleep.

The definitive event in OSA is occlusion of the upper airway usually at the level of the oropharynx. The resulting apnea leads to progressive asphyxia until there is a brief arousal from sleep, whereupon airway patency is restored and airflow resumes. The immediate factor leading to collapse of the upper airway in OSA is the generation of a critical subatmospheric pressure during inhalation that exceeds the ability of the airway dilator and abductor muscles to maintain airway stability.

The two major components of breathing are inhalation and exhalation.

Inhalation is an active process involving contraction of the diaphragm, external intercostal, and in certain circumstances, accessory muscles. It serves to increase intrathoracic volume, decrease intrapleural pressure and allow exchange of air and carbon dioxide within the alveoli of the lungs. Oxygen is transported from the alveoli to the pulmonary bloodstream by passive diffusion and is made available to tissues.

Exhalation, on the other hand, is a relatively passive process, requiring little or no contraction of the muscles during quiet breathing. A main function of the breathing process is to bring about the exchange of oxygen and carbon dioxide and other gaseous products from the biological system.

The opening of the upper airways is necessary in order to allow the passage of air since it is its only way in or out the body.

### Existing Solutions

Because the exact mechanism responsible for obstructive sleep apnea is not known, there is still no treatment that directly addresses the underlying problem.

### Pharmacologic Therapies

No medications are effective in the treatment of sleep apnea. However some physicians believe that mild cases of sleep apnea respond to drugs that either stimulate breathing or suppress deep sleep. Acetazolamide has been used to treat central apnea. Tricyclic antidepressants inhibit deep sleep, i.e. rapid eye movement (REM) state, and are useful only in subjects who have apneas in the REM state.

### Position Therapy

In mild cases of sleep apnea, breathing pauses occur only when the individual sleeps on the back. Thus using methods that will ensure that subjects sleep on their side are often helpful.

### Nasal Continuous Positive Airway Pressure (CPAP)

CPAP is the most common effective treatment for sleep apnea. In this procedure, the subject wears a mask or a pillow over the nose during sleep and pressure from an air compressor forces air through the nasal passages. The air pressure is adjusted so that it is just enough to hold the throat open when it relaxes the most. The pressure is constant and continuous. Nasal CPAP prevents obstruction while in use but apneas return when CPAP is stopped.

### Nocturnal Ventilation

Subjects can be ventilated non-invasively during sleep with positive pressure ventilation through a CPAP mask. This technique is now used in subjects whose breathing is impaired to the point that their blood carbon dioxide level is elevated, as happens in subjects with obesity-hypoventilation syndrome and certain neuromuscular disease.

### Dental Appliances

Dental appliances which reposition the lower jaw and the tongue have been helpful to some subjects with obstructive sleep apnea. Possible side effects include damage to teeth, soft tissues, and the jaw joint.

### Surgery

Some subjects with sleep apnea may require surgical treatment. Useful procedures include removal of adenoids and tonsils, nasal polyps or other growths, or other tissue in the airway, or correction of structural deformities. Younger subjects seem to benefit from surgery better than older subjects. However, surgical procedures are effective only 50 percent of the time because the exact location of the airway obstruction is usually unclear.

### Tracheotomy

Tracheotomy is used only in subjects with severe, life-threatening obstructive sleep apnea. In this procedure a small hole is made in the windpipe (trachea) below the Adam's apple. A T-shaped tube is inserted into the opening. This tube stays closed during waking hours and the person breathes normally. It is opened for sleep so that air flows directly into the lungs, bypassing any upper airway obstruction. Its major drawbacks are that it is a disfiguring procedure and the tracheotomy tube requires proper care to keep it clean.

### Uvulopalatopharyngoplasty (UPPP)

UPPP is a procedure used to remove excess tissue at the back of the throat (tonsils, adenoids, uvula, and part of the soft palate). This technique probably helps only half of the subjects who choose it. Its negative effects include nasal speech and backflow (regurgitation) of liquids into the nose during swallowing. UPPP is not considered as universally effective as tracheotomy but does seem to be a cure for snoring. It does not appear to prevent mortality form cardiovascular complications of severe sleep apnea.

Some subjects whose sleep apnea is due to deformities of the lower jaw (mandible) benefit from surgical advancement of the mandible. Gastric stapling procedures to treat obesity are sometimes recommended for sleep apnea subjects who are morbidly obese.

### SUMMARY

According to an illustrative embodiment of the present invention, there is provided a method of sensing the vagus nerve for the monitoring of respiratory activity.

According to a second illustrative embodiment of the present invention, there is provided a method of treating breathing disorders, such as, for example, sleep apnea.

In a third illustrative embodiment of the present invention, there is provided a system in the form of a generic bio-interfacing platform that may be adapted for either open-loop or closed-loop applications.

In an open-loop configuration, the bio-interfacing platform includes a sensing system directly interfaced with the peripheral nervous system with the aim of monitoring a physiological process such as, for example, the respiratory activity of a subject.

In a closed-loop configuration, the bio-interfacing platform includes sensing and stimulating systems directly interfaced with the peripheral nervous system and further includes at least one configurable implantable component that may be configured to implement any desired relationship between sensors (sensing system) and actuators (stimulating systems), with the aim of treating a disorder in a physiological process such as, for example, sleep apnea.

### BRIEF DESCRIPTION OF THE DRAWINGS

Non-limitative illustrative embodiments of the invention will now be described by way of example only with reference to the accompanying drawings, in which:

FIG. 1 is a flow diagram depicting the monitoring of respiratory activity process according to a first illustrative embodiment of the present invention;

FIG. 2 is a flow diagram depicting an example of a respiratory activity information extraction method to be used with the process of FIG. 1;

FIG. 3 is a graph of the estimation and measurement of flow and pressure during respiration at a slow rate;

FIG. 4 is a graph of the estimation and measurement of flow and pressure during respiration at a high rate;

FIG. 5 is a graph of the application of a matched filter to the data of FIG. 4 with a rectification and bin integration (RBI) bin size of 50 ms;

FIG. 6 is a flow diagram depicting the sleep apnea treatment process according to a second illustrative embodiment of the present invention;

FIG. 7 is a block diagram of the generic bio-interfacing platform according to a third illustrative embodiment of the present invention;

FIG. 8 is a block diagram of the bio-control unit (BCU) of the bio-interfacing platform of FIG. 7;

FIG. 9 is a block diagram of the bio-interfacing platform of FIG. 6 adapted for the monitoring of respiratory activity and treatment of sleep apnea;

FIG. 10 is a block diagram of the bio-control unit for sleep apnea (BCU-SA) of the bio-interfacing platform of FIG. 99;

FIG. 11 is a block diagram of the monitoring and apnea event detection module of the bio-control unit for sleep apnea (BCU-SA) of FIG. 10;

FIG. 12 is the graph of FIG. 4 illustrating the selection of offsets for controlling stimulation pacing;

FIG. 13 is a flow diagram depicting the process for providing stimulation pacing according to a third illustrative embodiment of the present invention;

FIG. 14 is a block diagram of the bio-control unit for stimulation pacing (BCU-P); and

FIG. 15 is a block diagram of the monitoring and pacing module of the bio-control unit for stimulation pacing (BCU-P) of FIG. 14.

### DETAILED DESCRIPTION

Generally stated, the non-limitative illustrative embodiment of the present invention provides a method and system for the monitoring of respiratory activity and further provides a method and system for the treatment of breathing disorders such as sleep apnea.

According to an illustrative embodiment of the present invention, there is provided a method of sensing the vagus nerve for the monitoring of respiratory activity.

According to a second illustrative embodiment of the present invention, there is provided a method of treating breathing disorders, such as, for example, sleep apnea.

In a third illustrative embodiment of the present invention, there is provided a system in the form of a generic bio-interfacing platform that may be adapted for either open-loop or closed-loop applications.

In an open-loop configuration, the bio-interfacing platform includes a sensing system directly interfaced with the peripheral nervous system with the aim of monitoring a physiological process such as, for example, the respiratory activity of a subject.

In a closed-loop configuration, the bio-interfacing platform includes sensing and stimulating systems directly interfaced with the peripheral nervous system and further includes at least one configurable implantable component that may be configured to implement any desired relationship between sensors (sensing system) and actuators (stimulating systems), with the aim of treating a disorder in a physiological process such as, for example, sleep apnea.

### Monitoring of Respiratory Activity

It has been discovered that the amplitude envelope of electroneurogram (ENG) signals recorded from the vagus nerve correlates well to respiratory activity.

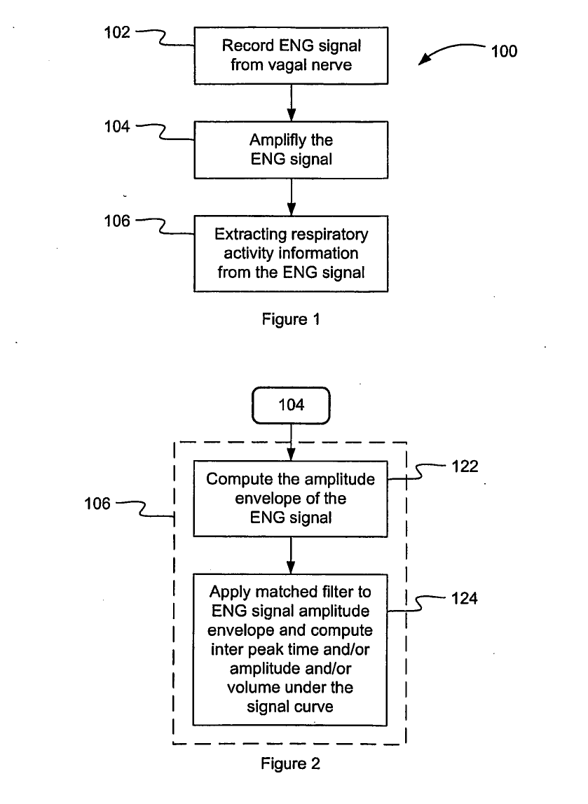

Referring to FIG. 1, there is shown in a flow diagram a process 100 for the monitoring of respiratory activity. The steps composing the process are indicated by blocks 102 to 106.

The process starts at block 102, where the ENG signal is recorded from the vagus nerve, after which, at block 104, the ENG signal is amplified.

Then, at block 106, respiratory activity information, such as, for example, respiratory flow, air volume and breathing intensity, is extracted from the ENG signal.

A method that may be used to obtain the respiratory activity information from the ENG signal at block 106 of process 100 is shown in FIG. 2. The sub-steps composing block 106 are indicated by blocks 122 an 124.

At block 122, the amplitude envelope of the amplified ENG signal is computed. The amplitude envelope may be computed by applying, for example, a rectification and bin integration (RBI) algorithm to the amplified ENG signal. This algorithm first rectifies the amplified ENG signal and sums the result in bins, essentially applying a low pass filter to the rectified signal.

A moving average filter may then be applied to the amplitude envelope, for example a moving average filter spanning second of data, and the result optimized using, for example, the solution to the Wiener-Hopf equation. The moving average filter helps to reduce the influence of variability inherent to ENG signals and its total length may be selected so as to be near the smallest feature (peak width) to be detected.

It is to be understood that other filtering solutions may be used for detecting respiratory activity from the ENG signal through combinations of algorithms that in essence implement rectification and some form of low-pass filtering (including matched filtering, simple averaging filters and finite-impulse-response filters). This will result in waveforms that may be used for subsequent peak detection.

Finally, at block 124, the respiratory flow inter peak time is computed, that is the time between successive inhalation/exhalation peaks, by detecting peaks associated with peak air outflow (exhalation) and peak air inflow (inhalation), and computing the time between each successive peaks. This may be accomplished by applying a matched filter amplitude envelope of the ENG signal in order to provide a signal on which flow peaks may be easily detected. The computed respiratory flow inter peak times may then be used to monitor the respiratory activity and may be, for example, displayed or provided to a further process or device. Optionally, the amplitude of the signal may also be computed and monitored so as to track changes in signal amplitude which are indicative of an increase in breathing intensity, i.e. increased pressure differential. Optionally still, the signal may also be integrated and monitored so as to track changes in air volume.

### Example

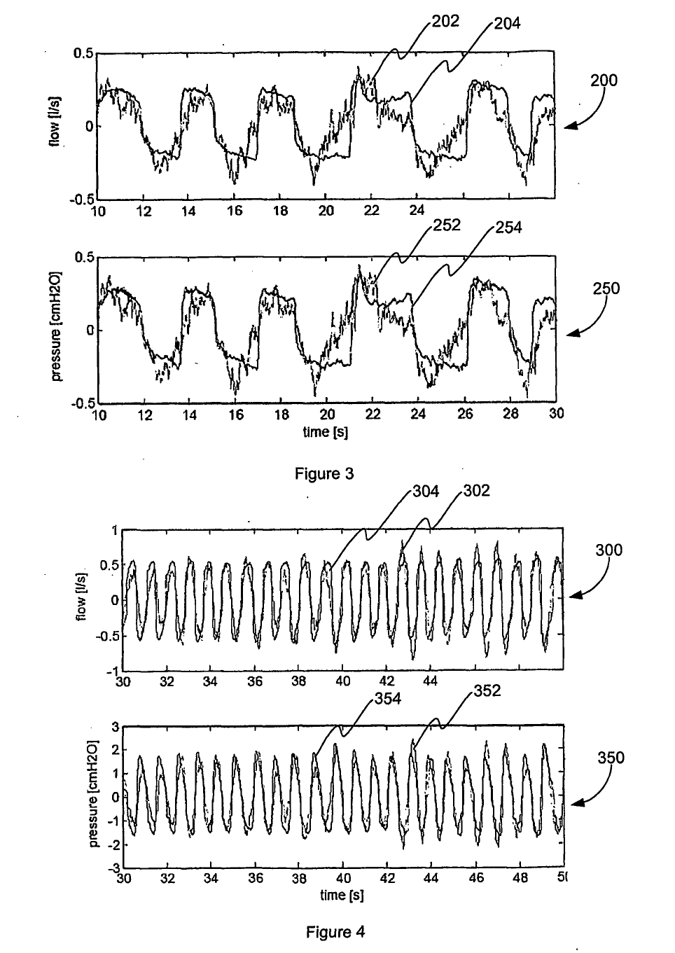

Referring to FIGS. 3 and 4, there are shown graphs of the estimation and measurement of flow 200, 300 and pressure 250, 300 during respiration at low 200, 250 and fast rates 300, 350, and demonstrate that the amplitude envelope of the ENG signal obtained from the vagus nerve may be used to monitor respiration.

The graphs 200, 250, 300 and 350 are obtained by first applying a RBI algorithm to the amplified ENG signal with a bin size of 10 ms in order to produce the amplitude envelope of the ENG signal and then applying a moving average filter spanning one second of data to the amplitude envelope of the ENG signal and optimizing the result.

More specifically, graph 200 of FIG. 3 shows the estimated respiratory flow 202 and the measured respiratory flow 204 versus time and graph 250 the estimated pressure 252 and the measured pressure 254 versus time during respiration at a low rate while graph 300 of FIG. 3 shows the estimated respiratory flow 302 and the measured respiratory flow 304 versus time and graph 350 the estimated pressure 352 and the measured pressure 354 versus time during respiration at a high rate.

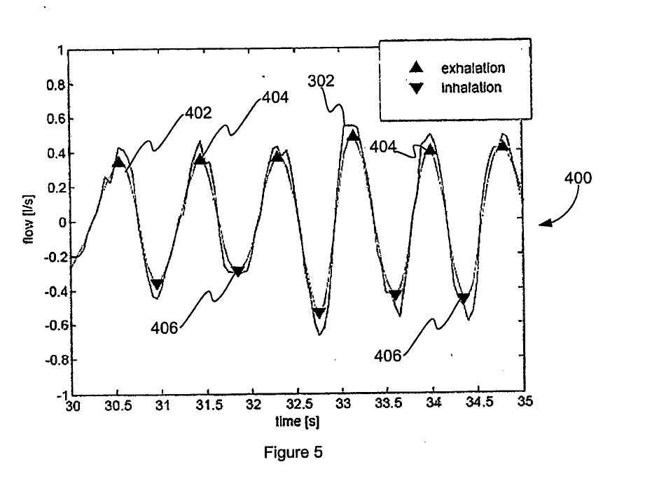

Referring now to FIG. 5, there is shown a graph 400 of the application of a matched filter to the estimated respiratory flow 302 of FIG. 4, but with a RBI bin size of 50 ms in order to produce a smooth output suitable for simple peak detection. This results in a reference signal 402 with positive peaks 404 representing exhalation and negative peaks 406 representing inhalation.

It is to be understood that although the method of computing the amplitude envelope will be used in the following description to describe the various illustrative embodiments, other methods exist for extracting information from the ENG signal may be used. For example methods based on non-rectified ENG and/or multi-channel ENG recordings, which may involve source separation techniques, feature extraction, and/or classification methods. Such methods may use the signal amplitude envelope as principal source of information, but may also use, for example, spectral characteristics (using wavelets for example) or features such as the number of zero-crossings per unit time, turns frequency, etc.

### Sleep Apnea

As previously explained, sleep apnea can cause excessive sleepiness and may lead to further health problems in the long term. Research on the relationship between vagus nerve activity and respiration has demonstrated that the vagus nerve ENG signal may be used to detect sleep apnea events.

Accordingly, based on the ability to monitor respiratory activity using a ENG signal recorded on the vagus nerve, as described, for example, by process 100 of FIG. 1, it is possible to detect sleep apnea events.

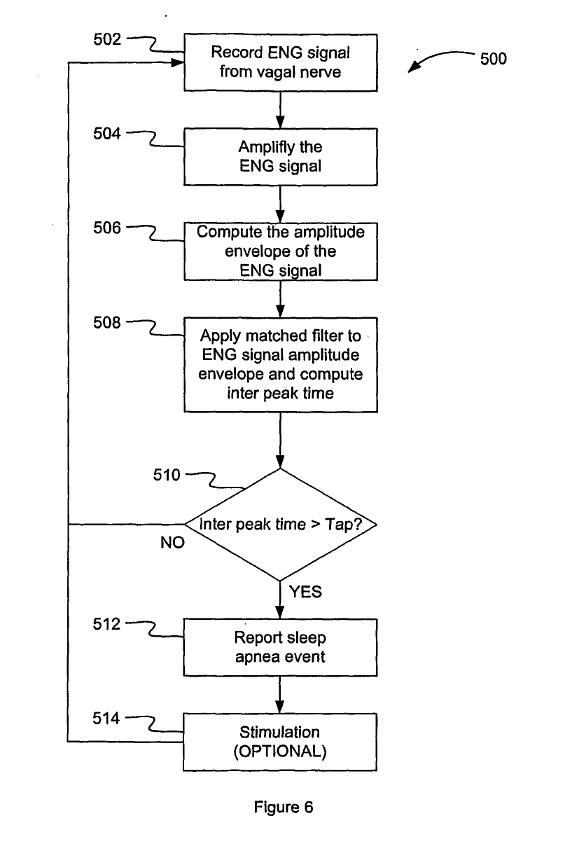

Referring to FIG. 6, there is shown in a flow diagram a process 500 for the detection of sleep apnea events. The steps composing the process are indicated by blocks 502 to 514.

For concision purposes, only blocks 510 to 514 will be described as blocks 502 to 508 have already been described in process 100 of FIG. 1 as block 102 to 106, with blocks 506 and 508 being detailed in FIG. 2 as block 122 and 124.

At block 510, the process 500 verifies if the respiratory flow inter peak time is greater than Tₐₚ. In the case of a sleep apnea event, the airway is blocked during inhalation, resulting in either a delayed peak in inhalation flow, i.e. a negative peak or else a delayed peak in exhalation flow, i.e. positive peak, if a sufficiently strong inhalation occurred before the apnea event. Accordingly, Tₐₚ may be set to, for example, seven seconds, which is a little below the 10 second interval at which an obstruction is considered an apnea event. However, the value of Tₐₚ may need to be set individually for each subject and depends on the normal respiration rate during sleep.

If the respiratory flow inter peak time is not greater than Tₐₚ, the process 500 proceeds back to block 502 where the recording of the ENG signal continues.

If the respiratory flow inter peak time is greater than Tₐₚ, the process 500 proceeds to block 512 where the sleep apnea event is reported. Optionally, at block 514, airway opening stimulation may be triggered in response to the detection of the sleep apnea event.

The airway opening stimulation may take a number of different forms depending on the type of sleep apnea, i.e. central (CSA), obstructive (OSA) and mixed.

For example, for cases of obstructive sleep apnea (OSA), possible targets for stimulation include the genioglossus muscle, which moves the tongue forward in the mouth and opens the upper airway and/or the hypoglossal nerve which innervates the genioglossus muscle, and/or other parts of the nervous system which result in increased tonicity in the genioglossus muscle and/or other muscles that open the airway.

As for cases of central sleep apnea (CSA), phrenic nerve pacing may also be used, which could help the subject to breath during its sleep. Phrenic nerve pacing stimulates the nerve to allow the diaphragm to contract (inhalation) and stops stimulating for the muscle to relax (exhalation). Alternatively, the muscles of the diaphragm may be stimulated directly to the same effect.

For cases of mixed OSA and CSA, the CSA approach may be used, as the subsequent obstruction may be due to an absence of air flow to trigger the negative pressure reflex that assists in maintaining airway patency. Should this prove insufficient, the methods for CSA and OSA as described above may be combined, involving stimulation to promote airway patency as well as expansion of lung volume.

The process 500 then proceeds back to block 502 where the recording of the ENG signal continues.

### Stimulation During Each Inhalation

A technically simple, and therefore very robust, way of treating OSA, or other breathing disorders, is to use the monitoring of the respiratory activity in order to provide stimulation pacing. In this scheme the airways are stimulated during each inhalation in order to ensure airway patency. The targets are identical to those listed above for OSA.

Basically, the muscles that maintain airway patency are stimulated during each inhalation; during exhalation the stimulation is turned off. The measured respiration signal indicates the respiratory rhythm and when inhalation starts.

Referring to FIG. 12, the time to start stimulation is defined as the moment of peak exhalation 902 plus a first offset Δ1, whose value should be selected such that stimulation starts just before the next inhalation 904 starts. The time to stop stimulation is defined as the moment of peak inhalation 904 plus a second offset Δ2, whose value should be selected such that stimulation stops near the end of inhalation, i.e. the next exhalation 906 starts. The offsets Δ1 and Δ2 may be adjusted dynamically based on the observed respiration rhythm.

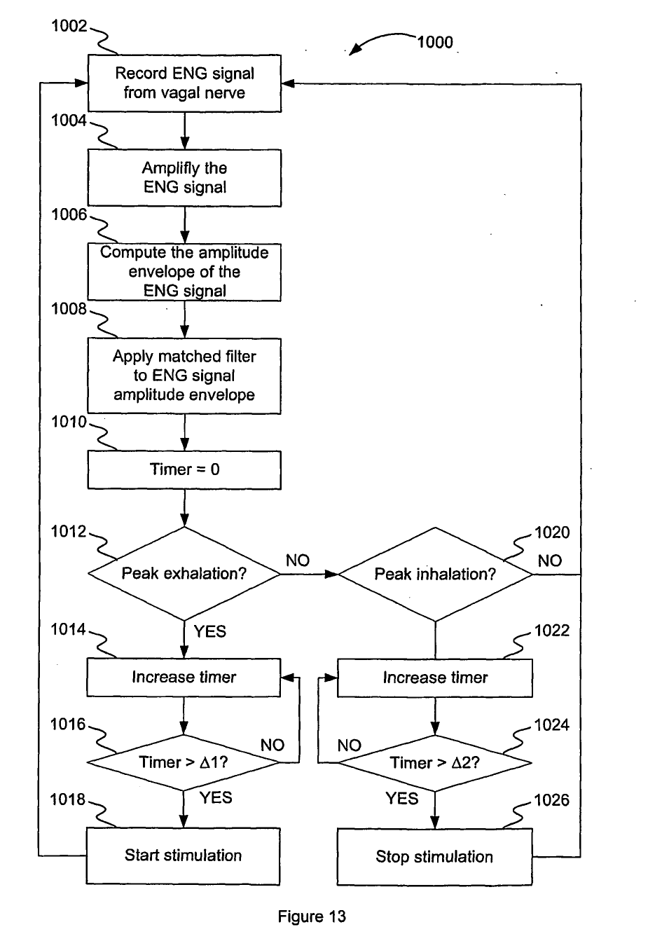

Referring now to FIG. 13, there is shown in a flow diagram a process 1000 for providing stimulation pacing. The steps composing the process are indicated by blocks 1002 to 1026.

For concision purposes, only blocks 1010 to 1026 will be described as blocks 1002 to 1008 have already been described in process 100 of FIG. 1 as block 102 to 106, with blocks 1006 and 1008 being detailed in FIG. 2 as block 122 and 124, with the exception that at block 1008.

At block 1010, the process 1000 initiates a timer and, at block 1012, verifies if it detects a positive peak, i.e. a peak associated with peak air outflow (exhalation). If so, it proceeds to block 1014, if not, to block 1020.

At block 1014, the timer is increased and, at block 1016, the process 1000 verifies if the timer is greater than the first offset Δ1. If the timer is greater than the first offset Δ1, the process 1000 proceeds to block 1018, where the stimulation is started, and then proceeds back to block 1002. If not, it proceeds back to block 1016.

At block 1020, the process 1000 verifies if it detects a negative peak, i.e. a peak associated with peak air inflow (inhalation). If so, it proceeds to block 1022, if not, it proceeds back to block 1002.

At block 1022, the timer is increased and, at block 1024, the process 1000 verifies if the timer is greater than the second offset Δ2. If the timer is greater than the second offset Δ2, the process 1000 proceeds to block 1026, where the stimulation is stopped, and then proceeds back to block 1002. If not, it proceeds back to block 1022.

### Hypopnea Detection

The ideal solution for the treatment of sleep apnea intervenes only when necessary and before the airway obstruction occurs. The onset of a sleep apnea event is characterized by a narrowing airway and a concomitant increase in the pressure differential between the lungs and the ambient pressure. This may be characterized as hypopnea, or a reduced capacity to breathe.

The ENG signal recorded from the vagus nerve may be used to detect hypopnea in order to trigger a stimulation before an obstruction of the airways occurs. When hypopnea occurs there is an increased effort in breathing, which is reflected in the sensory feedback as an increase in the amplitude envelope of the amplified ENG signal (from mechanical stretch receptors in the lungs).

Referring back to FIG. 6, the process 500 used for the detection of sleep apnea events may be modified so as to detect hypopnea. In this regard, at blocks 508 and 510, instead of computing the respiratory flow inter peak time and verifying if it is greater than Tₐₚ, a predictor which uses previous respiration activity to predict the current behavior may be used to monitor for deviations between predicted and actual behavior to determine whether there is an increased effort being made. The increased effort is expected to be reflected in an increased amplitude and, as a secondary characteristic, an increase in breathing rhythm.

### Generic Bio-Interfacing Platform

The generic bio-interfacing platform is a general-purpose platform with an architecture design that lends itself to easy extension of capabilities without a complete redesign. This implies a very modular design approach where functional units are identified as modules and are primarily specified with the characteristics of their associated inputs and outputs. Advantageously, to reduce design efforts, the bio-interfacing platform is also scalable, meaning that the capacity of the design may be extended by replicating modules. This means that the modules are designed with parallel operation in mind. Two general frameworks may be adopted: a “star-topology” where parallel modules connect to a hub, and “full-parallel” where modules cooperate through a system bus.

The modules composing the bio-interfacing platform are of two types: modules that have generic functions and are common to the various applications, and modules which are implementation specific and may vary from one application to another.

Thus, “general-purpose” in the context of the generic bio-interfacing platform implementation means that the platform includes a suitable core set of modules with which systems for various applications may be developed, either in an open-loop or closed-loop configuration. For example, it may be possible to use the same core set of modules for both a system to control urinary incontinence, for regulation of insulin release or treating sleep apnea by changing the application specific modules of the generic bio-interfacing platform.

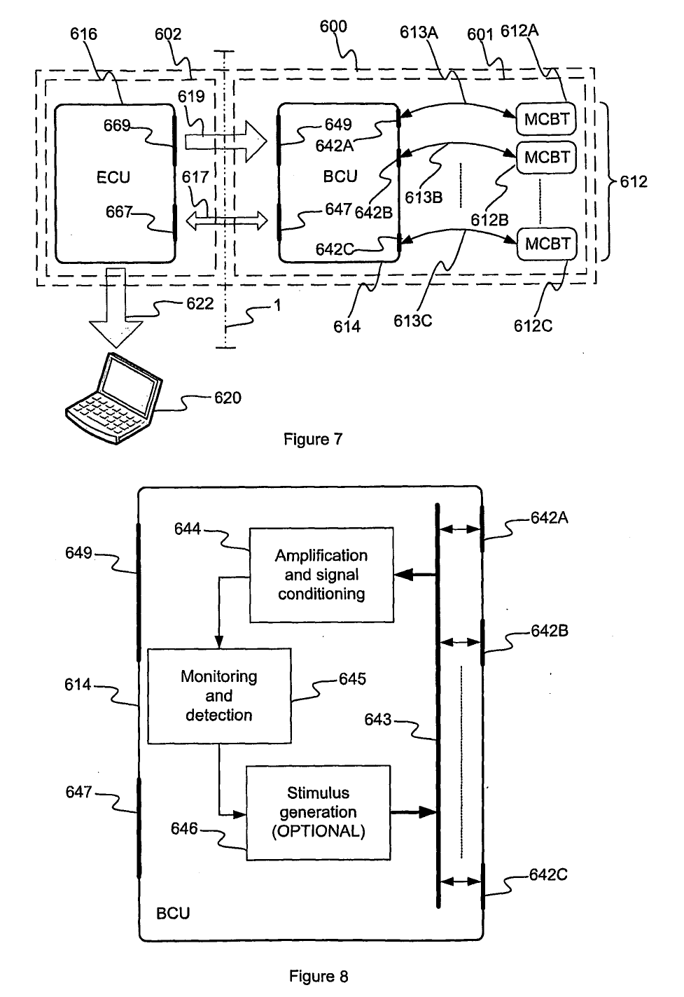

Referring to FIG. 7, there is shown a block diagram of the generic bio-interfacing platform 600 having an implantable portion 601, which includes multi-channel bio-transducers (MCBT) 612A, 612B, 612C, connected to at least one bio-control unit (BCU) 614 through respective leads or wireless links 613A, 613B, 613C, and BCU connectors 642A, 642B, 642C, and an external portion 602, which includes an external control unit (ECU) 616. The BCU 614 and ECU 616 may communicate with each other using a communication link 617 across the skin 1 using respective transceivers 647 and 667.

### MCBT

The MCBT, collectively identified by numeral 612, include sensors and actuators used to record/sense, actuate/stimulate or both.

Sensors may include one or more of the following: a pressure sensor, a temperature sensor, a thoracic impedance sensor, a heart rate sensor, an acoustic sensor, a kinematic sensor, a kinetic sensor, a myoelectric sensor, a neuro sensor, an electrode, a probe, etc. It is to be understood that other types of sensors may be used.

Actuators may include one or more of the following: a muscle stimulation electrode (for example an epimysial muscle stimulation electrode), a drug pump, a mechanical actuator, an acoustic actuator, etc. It is to be understood that other types of actuators may be used.

It is further to be understood that the number and types of sensors and/or actuators may vary depending on the application and that multiple sensors and/or actuators of the same type, or combinations thereof, may be used. It is also to be understood that the sensors and/or actuators may be implantable et externally positioned.

In the illustrative embodiments, the MCBT 612 includes a cuff adapted to surround part of a nerve and provided with multiple chambers, for example four, having therein electrodes, to provide recording/sensing and/or actuating/stimulation selectivity around the nerve surface. Furthermore, in order to increase sensitivity, the electrodes may be in a tri-polar configuration and designed so as to be created from a continuous wire without any soldering.

An example of a device that may be used as a MCBT 612 is the cuff-electrode, which is a transducer that may be used to both measure peripheral nerve signals and stimulate peripheral nerve activity. An example of a cuff-electrode that may be used is disclosed in U.S. Pat. No. 5,824,027 entitled “NERVE CUFF HAVING ONE OR MORE ISOLATED CHAMBERS”, issued Oct. 20, 1998, to Hoffer et al. It is to be understood that other types of electrodes, leads, probes, cuff-electrodes, etc., may be used as well. Other examples of cuff electrodes that may be used are disclosed in PCT patent application No. PCT/CA2007/000991 entitled “NERVE CUFF, METHOD AND APPARATUS FOR MANUFACTURING SAME”, filed Jun. 4, 2007, by Hoffer et al. and PCT patent application No. ______ entitled “NERVE CUFF INJECTION MOLD AND METHOD OF MAKING A NERVE CUFF”, filed Aug. 29, 2007, by Imbeau et al.

### BCU

Referring to FIG. 8, there is shown a block diagram of the BCU 614, which implements the core functionality of the generic bio-interfacing platform 600. The main components of the BCU 614 are the connectors 642A, 642B, 642C, for connecting the leads 613A, 613B, 613C of MCBT 612A, 612B, 612C respectively, an amplification and signal conditioning module 644 for processing signals coming from the MCBT 612, a monitoring and detection module 645, which monitors one or more physiological process and may detect if a deficiency condition is present, an optional stimulus generation module 646 for generating one or more actuation/stimulation signal aimed at specific MCBT 612 in order to correct the deficiency condition identified by the monitoring and detection module 645, and a data bus 43 allowing the exchange of signals between the individual MCBT 612A, 612B, 612C and both the amplification and signal conditioning module 644 and the stimulus generation module 646.

Examples of connectors that may be used for connectors 642A, 642B, 642C are disclosed in U.S. patent application Ser. No. 10/861,323 entitled “IMPLANTABLE MODULAR MULTI-CHANNEL CONNECTOR SYSTEM FOR NERVE SIGNAL SENSING” filed Jun. 3, 2004, by Hoffer et al. and PCT patent application No. ______ entitled “HIGH DENSITY IMPLANTABLE CONNECTOR”, filed Aug. 28, 2007, by Richard et al.

The signal conditioning module 644 may include, without limiting the illustrative embodiment to these components, an ENG signal amplifier and a rectifier circuit. Examples of amplifiers and rectifier circuit that may be used are respectively disclosed in U.S. patent application Ser. No. 11/315,884 entitled “IMPLANTABLE SIGNAL AMPLIFYING CIRCUIT FOR ELECTRONEUROGRAPHIC RECORDING”, filed Dec. 21, 2005, by Baru Fassio and U.S. patent application Ser. No. 10/935,699 entitled “PRECISION RECTIFIER CIRCUIT FOR CHANNEL CONNECTOR SYSTEM FOR NERVE SIGNAL SENSING”, filed Sep. 7, 2004, by Baru Fassio.

The monitoring and detection module 645 is a non-generic module that contains software that makes each BCU 614 an application specific module, thus, advantageously, the monitoring and detection module 645 may be implemented using a microcontroller, so that different applications require only adaptation of the software.

As mentioned previously, the BCU 614 also includes a transceiver 647 for providing communication between the BCU 614 and the ECU 616.

The BCU 614 power source may either be a built-in permanent battery or may be a rechargeable battery which is replenished using power transfer across the skin 1 between optional BCU 614 power input 649 and ECU 616 power output 669 using, for example, but not limiting the illustrative embodiment to this specific example, a RF magnetic field 619. In an alternative embodiment, the BCU 614 may not include any power source at all and run directly on power transmitted by the ECU 616 through the skin 1 using the power input 649 and the power output 669 as power interfaces.

Optionally, the generic bio-interfacing platform 600 may include a transcutaneous energy transfer system (TETS) between the ECU 616 and the BCU 614 involving feedback through the communication link 617, which allows regulation of the power transfer based on power need during the charge process.

It is to be understood that the number of BCU 614 may vary depending on the application.

### ECU

The ECU 616 is a device which may be used by, for example, a practitioner or a subject to interact with a BCU 614 through a two-way communication link 617. For example, the clinician monitoring the subject may use the ECU 616 to ensure an implant incorporating an application specific bio-interfacing platform is functioning correctly and perhaps monitor physiological processes, retrieve status information or to control a specific BCU 614. Also, a subject may use the ECU 616 to access basic status information such as, for example, system integrity or battery status, or to initiate an exercise program.

Optionally, the ECU 616 may also provide, through a wireless or wired communication link 622, for example through a USB port, remote monitoring of the BCU 614 through, for example, a personal digital assistant (PDA) or personal computer (PC) 620. Furthermore, the ECU 616 may also include a power output 669 as previously discussed.

### Bio-Interfacing Platform for the Monitoring of Respiratory Activity and Treatment of Sleep Apnea

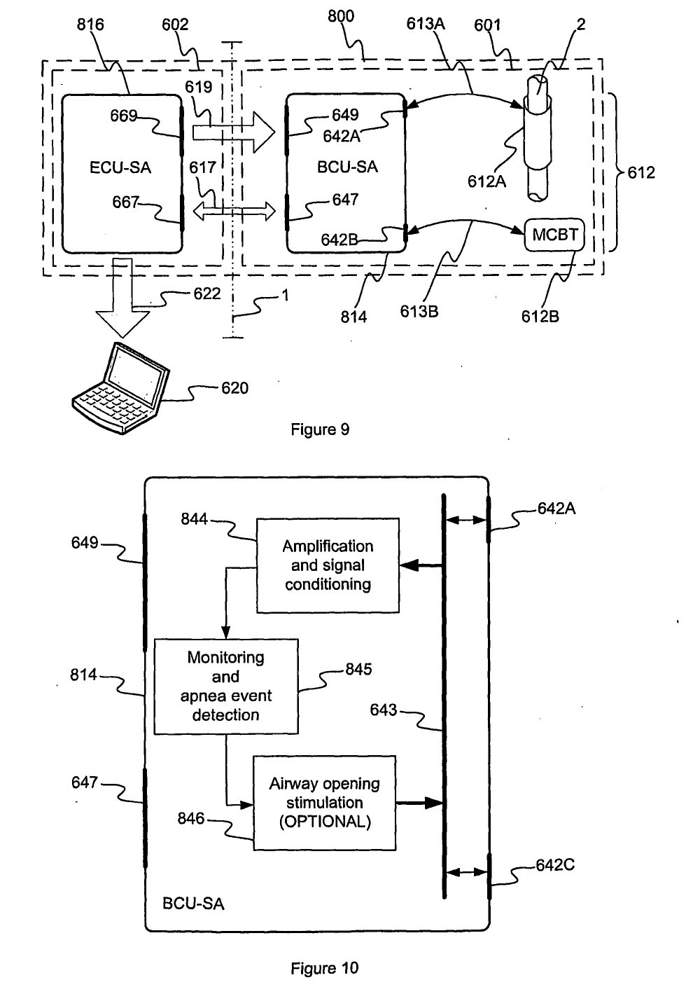

Referring to FIG. 9, there is shown a block diagram of an example of an advanced neuromodulator 800 for the monitoring of respiratory activity and, optionally, the treatment of sleep apnea based on the generic bio-interfacing platform 600 of FIG. 7.

A first MCBT 612A in the form of a cuff electrode is placed around the vagus nerve 2 in order to record ENG signals from the subject. A suitable location for placement of the cuff electrode 612A may be, for example, in the neck, but other locations along the vagus nerve between the head and pulmonary branches of the subject may be considered. A lead 613A connects the cuff electrode 612A to the BCU-SA 814.

It is to be understood that the BCU-SA 814 and the ECU-SA 816 refer to the generic BCU 614 and ECU 616 of the generic bio-interfacing platform 600 that have been adapted for the monitoring of respiratory activity and, optionally, the treatment of sleep apnea.

Referring now to FIG. 10, there is shown a block diagram of the BCU-SA 814, which includes an amplification and signal conditioning module 844, a monitoring and apnea event detection module 845 and an optional airway opening stimulation module 846.

The amplification and signal conditioning module 844 amplifies the ENG signal recorded by the cuff electrode 612A and provides the amplified ENG signal to the monitoring and apnea event detection module 845, which includes an algorithm that uses the amplified ENG to monitor respiratory activity and, optionally, detect apnea events before they result in arousal from sleep. The algorithm executed by the monitoring and apnea event detection module 845 implements blocks 506 to 512 of process 500 shown in FIG. 6. Optionally, upon the detection of an apnea event, the monitoring and apnea event detection module 845 may send a trigger to the optional airway opening stimulation module 846, which causes the airway to open through stimulation using MCBT 612B.

As previously mentioned, for cases of obstructive sleep apnea (OSA), possible targets for stimulation provided by MCBT 612B include the genioglossus muscle and/or the hypoglossal nerve and/or other parts of the nervous system which result in increased tonicity in the genioglossus muscle and/or other muscles that open the airway.

As also previously mentioned, for cases of central sleep apnea (CSA), the stimulation provided by MCBT 612B may be used for phrenic nerve pacing, which could help the subject to breath during its sleep.

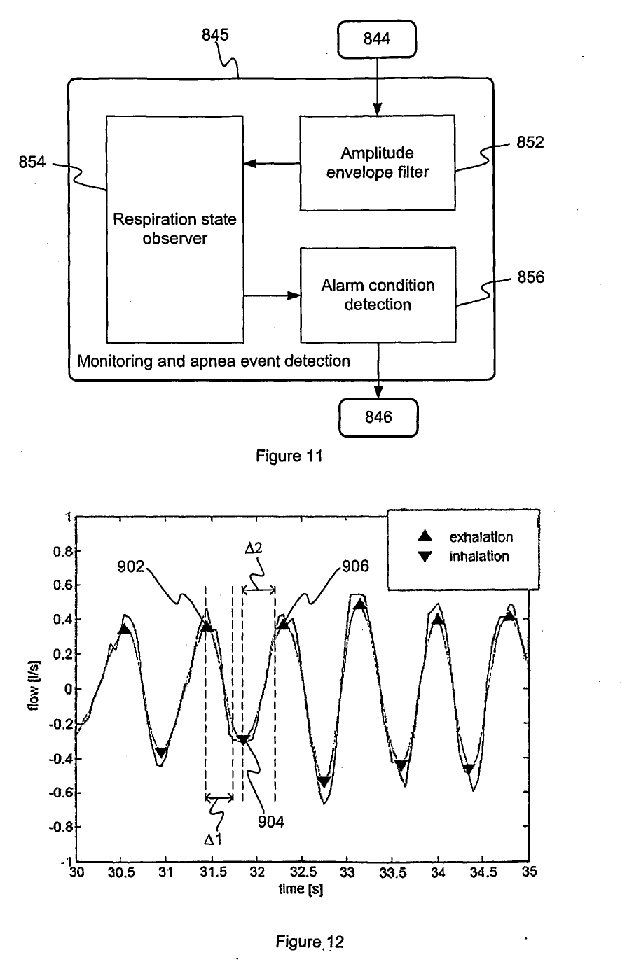

Referring now to FIG. 11, there is shown a block diagram of the monitoring and apnea event detection module 845 discussed above, which includes an amplitude envelope filter sub-module 852, a respiration state observer sub-module 854 and an alarm condition detection sub-module 856.

The amplitude envelope filter sub-module 852 produces an amplitude envelope of the amplified ENG signal, provided by the amplification and signal conditioning module 844, by implementing block 506 of process 500.

The respiration state observer sub-module 854 detects, in the amplitude envelope of the ENG signal obtained from the vagus nerve 2, peaks associated with peak air outflow and peak air inflow, and reports the time between each successive peaks, by implementing block 508 of process 500.

Finally, the alarm condition detection sub-module 856, using the time between each successive peaks, verifies if a sleep apnea event is present, and if so reports it using transceiver 647 and by implementing blocks 510 and 512 of process 500. The reporting of the sleep apnea event may be effectuated by the ECU-SA 816 receiving the indication of a sleep apnea event from the BCU-SA 814 though its transceiver 667 and communication link 617. Optionally, the reporting of the sleep apnea event may further be provided to a PDA or PC 620 receiving indication of the sleep apnea event from the ECU-SA 816 through communication link 622. It is to be understood that the peak air outflow and peak air inflow, as well as inter peak time, may also be similarly reported for continuous monitoring of the respiratory activity of the subject.

Optionally, if a sleep apnea event is detected, the alarm condition detection sub-module 856 may send a trigger the optional airway opening stimulation module 846 as previously mentioned.

### Bio-Interfacing Platform for the Monitoring of Respiratory Activity and Stimulation Pacing

The advanced neuromodulator 800 of FIG. 9 may be modified so as to provide stimulation pacing by replacing the BCU-SA 814 with a BCU-P 1114, which refers to the generic BCU 614 of the generic bio-interfacing platform 600 of FIG. 6 that has been adapted for the monitoring of respiratory activity and stimulation pacing.

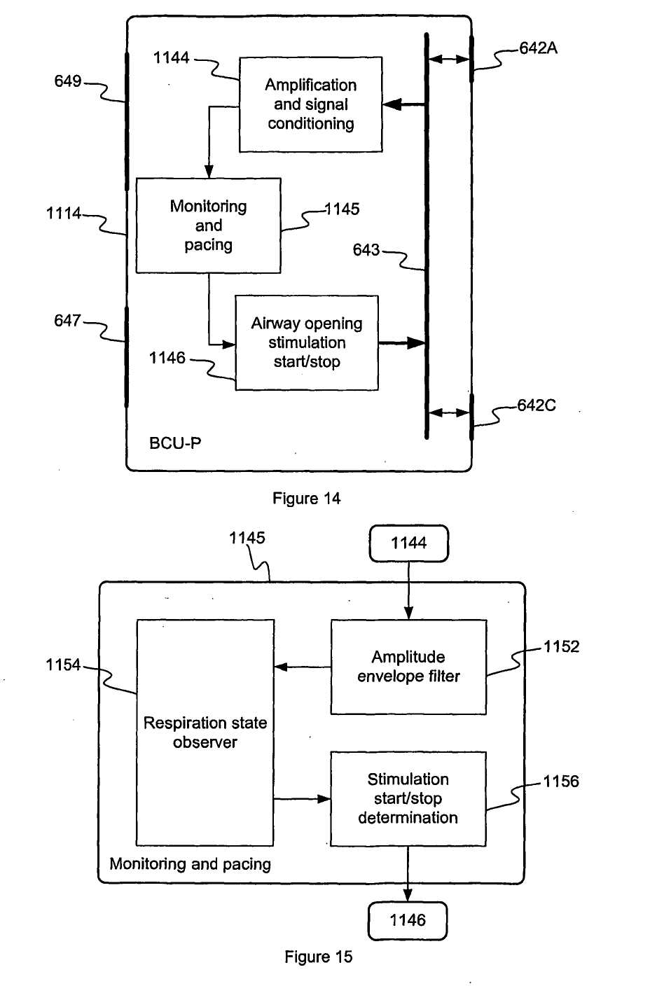

Referring to FIG. 14, there is shown a block diagram of the BCU-P 1114, which includes an amplification and signal conditioning module 1144, a monitoring and pacing module 1145 and an airway opening stimulation start/stop module 1146.

The amplification and signal conditioning module 1144 amplifies the ENG signal recorded by the cuff electrode 612A and provides the amplified ENG signal to the monitoring and pacing module 1145, which includes an algorithm that uses the amplified ENG to monitor respiratory activity and start or stop airway opening stimulation in order to maintain airway patency. The algorithm executed by the monitoring and pacing module 1145 implements blocks 1006 to 1016 and 1020 to 1024 of process 1000 shown in FIG. 12. Upon determination that stimulation should be started or stopped, the monitoring and pacing module 1145 sends a trigger to the airway opening stimulation start/stop module 1146, which initiates or ceases stimulation using MCBT 612B.

As previously mentioned, possible targets for stimulation provided by MCBT 612B include the genioglossus muscle and/or the hypoglossal nerve and/or other parts of the nervous system which result in increased tonicity in the genioglossus muscle and/or other muscles that open the airway.

Referring now to FIG. 15, there is shown a block diagram of the monitoring and pacing module 1145 discussed above, which includes an amplitude envelope filter sub-module 1152, a respiration state observer sub-module 1154 and stimulation start/stop determination sub-module 1156.

The amplitude envelope filter sub-module 1152 produces an amplitude envelope of the amplified ENG signal, provided by the amplification and signal conditioning module 1144, by implementing block 1106 of process 1000.

The respiration state observer sub-module 1154 detects, in the amplitude envelope of the ENG signal obtained from the vagus nerve 2, peaks associated with peak air outflow and peak air inflow by implementing block 1008 of process 1000.

Finally, the stimulation start/stop determination sub-module 1156 verifies if stimulation is to be initiated or ceased by implementing blocks 1010 to 1016 and 1020 to 1024 of process 1000 and accordingly sends a trigger the airway opening stimulation start/stop module 1146 as previously mentioned.

It is to be understood that the various units, modules and sub-modules and algorithms may be implemented using, for example one or more electronic circuit, microcontroller or DSP.

It is also to be understood that the various illustrative embodiments of processes and bio-interfacing platform for the detection of sleep apnea, or other breathing deficiencies, may be selectively activated, for example when a subject is sleeping. The activation may be user initiated, optionally with a delay, according to a given schedule, by monitoring the heart rate of the subject, the orientation of the subject, etc.

Although the present invention has been described by way of illustrative embodiments and examples thereof, it should be noted that it will be apparent to persons skilled in the art that modifications may be applied to the present particular embodiment without departing from the scope of the present invention.

What is claimed is:

1. A method for monitoring the respiratory activity of a subject, comprising the steps of:
recording an electroneurogram signal from the vagus nerve of the subject;
amplifying the electroneurogram signal;
computing an amplitude envelope of the amplified electroneurogram signal;
applying a matched filter to the amplitude envelope; and
computing a time between successive peaks of the filtered amplitude envelope;
wherein the time between successive peaks is indicative of the respiratory activity of the subject.

2. A method for monitoring the respiratory activity of a subject, comprising the steps of:
recording an electroneurogram signal from the vagus nerve of the subject;
amplifying the electroneurogram signal;
computing an amplitude envelope of the amplified electroneurogram signal;
applying a matched filter to the amplitude envelope; and
computing a time between successive peaks of the filtered amplitude envelope;
wherein the time between successive peaks is indicative of the respiratory activity of the subject.

3. A method according to claim 1, further comprising the step of displaying the time between successive peaks.

4. A method according to claim 1, wherein the electroneurogram signal is recorded from a portion of the vagus nerve located between the head and the pulmonary branches of the subject.

5. A method according to claim 1, wherein the electroneurogram signal is recorded from a portion of the vagus nerve located in the neck of the subject.

6. A method according to claim 1, wherein the amplitude envelope computing step includes includes the application of a low-pass filter.

7. A method according to claim 1, wherein the amplitude envelope computing step includes applying a filter selected from a group consisting of a matched filter, a simple averaging filter and a finite-impulse-response filter.

8. A method according to claim 1, wherein the amplitude envelope computing step includes includes applying a rectification and bin integration algorithm to the amplified electroneurogram signal.

9. A method according to claim 8, wherein the amplitude envelope computing step includes further includes applying a moving average filter to the amplified electroneurogram signal after the application of the rectification and bin integration algorithm.

10. A method according to claim 9, wherein the amplitude envelope computing step includes further includes optimizing the result of the moving average filter using the solution to the Wiener-Hopf equation.

11. A method according to claim 1, further comprising the steps of:
comparing the time between successive peaks with a sleep apnea event threshold;
reporting a sleep apnea event should the compared time between successive peaks be greater than the sleep apnea event threshold.

12. A method according to claim 11, wherein the apnea event threshold is set individually for each subject according to the normal respiration rate of the subject during sleep.

13. A method according to claim 11, wherein the apnea event threshold is set to about 10 seconds.

14. A method according to claim 11, wherein the reporting step includes triggering an airway opening stimulation.

15. A method according to claim 14, wherein the airway opening stimulation includes stimulation of the genioglossus muscle.

16. A method according to claim 14, wherein the airway opening stimulation includes stimulation of the hypoglossal nerve.

17. A method according to claim 14, wherein stimulation is applied to the phrenic nerve in order to maintain respiration.

18. A method for maintaining airway patency of a subject through stimulation, comprising the steps of:
recording an electroneurogram signal from the vagus nerve of the subject;
amplifying the electroneurogram signal;
computing an amplitude envelope of the amplified electroneurogram signal;
applying a matched filter to the amplitude envelope;
detecting a positive peak in the filtered amplitude envelope;
waiting for a duration equal to a first offset value;
triggering an airway opening stimulation;
detecting a negative peak in the filtered amplitude envelope;
waiting for a duration equal to a second offset value; and
stopping the airway opening stimulation.

19. A method according to claim 18, wherein the first and second offset values are computed from a previously obtained respiratory rhythm.

20. A method according to claim 18, wherein the electroneurogram signal is recorded from a portion of the vagus nerve located between the head and the pulmonary branches of the subject.

21. A method according to claim 18, wherein the electroneurogram signal is recorded from a portion of the vagus nerve located in the neck of the subject.

22. A method according to claim 18, wherein the airway opening stimulation includes stimulation of the genioglossus muscle.

23. A method according to claim 18, wherein the airway opening stimulation includes stimulation of the hypoglossal nerve.

24. A method according to claim 18, wherein the amplitude envelope computing step includes the application of a low-pass filter.

25. A method according to claim 18, wherein the amplitude envelope computing step includes applying a filter selected from a group consisting of a matched filter, a simple averaging filter and a finite-impulse-response filter.

26. A method according to claim 18, wherein the amplitude envelope computing step includes applying a rectification and bin integration algorithm to the amplified electroneurogram signal.

27. A method according to claim 26, wherein the amplitude envelope computing step further includes applying a moving average filter to the amplified electroneurogram signal after the application of the rectification and bin integration algorithm.

28. A method according to claim 27, wherein the amplitude envelope computing step further includes optimizing the result of the moving average filter using the solution to the Wiener-Hopf equation.

29. A method for detecting hypopnea during the respiratory activity of a subject, comprising the steps of:
recording an electroneurogram signal from the vagus nerve of the subject;
amplifying the electroneurogram signal;
computing an amplitude envelope of the amplified electroneurogram signal;
applying a matched filter to the amplitude envelope;
computing a deviation of the signal resulting from the application of the matched filter to the amplitude envelope from a predictor, the predictor being based on previously obtained respiration activity;
reporting a hypopnea event should the deviation be greater than a hypopnea event threshold.

30. A method according to claim 29, wherein the deviation is an increase in amplitude.

31. A method according to claim 29, wherein the deviation is an increase in respiratory rhythm.

32. A method according to claim 29, wherein the electroneurogram signal is recorded from a portion of the vagus nerve located between the head and the pulmonary branches of the subject.

33. A method according to claim 29, wherein the electroneurogram signal is recorded from a portion of the vagus nerve located in the neck of the subject.

34. A system for monitoring the respiratory activity of a subject, comprising:
an electrode for detecting an electroneurogram signal from the vagus nerve of the subject;
a transceiver;
an implantable control unit operatively connected to to the electrode and the transceiver, the implantable control unit including:
a signal amplifier for amplifying the electroneurogram signal;
a rectifier for rectifying the amplified electroneurogram signal;
a monitoring and detection module for:
computing an amplitude envelope of the amplified electroneurogram signal;
applying a matched filter to the amplitude envelope; and
computing a time between successive peaks from filtered amplitude envelope; and
transmitting the computed time between successive peaks using the transceiver;
wherein the time between successive peaks is indicative of the respiratory activity of the subject.

35. A system according to claim 34, wherein the electrode includes a cuff electrode assembly adapted to surround part of the vagus nerve of the subject.

36. A system according to claim 35, wherein the cuff electrode assembly is provided with multiple chambers having electrodes therein.

37. A system according to claim 34, further comprising an external control unit including a transceiver for communication with the transceiver of the implantable control unit, the external control unit allowing interaction with the implantable control unit.

38. A system according to claim 37, wherein the external and implantable control units further include respective power interfaces for transferring power from the external control unit to the implantable control unit.

39. A system according to claim 34, wherein the implantable control unit further includes a power source.

40. A system according to claim 34, wherein the algorithm further includes:
comparing the time between successive peaks with a sleep apnea event threshold;
transmitting the occurrence of a sleep apnea event using the transceiver should the compared time between successive peaks be greater than the sleep apnea event threshold.

41. A system according to claim 40, wherein the apnea event threshold is set individually for each subject according to the normal respiration rate of the subject during sleep.

42. A system according to claim 40, further comprising a second electrode and wherein the algorithm further includes triggering an airway opening stimulation using the second electrode.

43. A system according to claim 42, wherein the second electrode is configured to be positioned in contact with the genioglossus muscle.

44. A system according to claim 42, wherein the second electrode is configured to be positioned in contact with the genioglossal nerve.

45. A system according to claim 42, wherein the second electrode is configured to be positioned in contact with the phrenic nerve.

46. A system for maintaining airway patency of a subject through stimulation, comprising:
a first electrode for recording an electroneurogram signal from the vagus nerve of the subject;
a second electrode;
a transceiver;
an implantable control unit operatively connected to the first and second electrodes and to the transceiver, the implantable control unit including:
a signal amplifier for amplifying the electroneurogram signal;
a rectifier for rectifying the amplified electroneurogram signal;
a monitoring and detection module for:
computing an amplitude envelope of the amplified electroneurogram signal;
applying a matched filter to the amplitude envelope;
detecting a positive peak in the filtered amplitude envelope;
waiting for a duration equal to a first offset value;
triggering an airway opening stimulation using the second electrode;
detecting a negative peak in the filtered amplitude envelope;
waiting for a duration equal to a second offset value; and
stopping the airway opening stimulation.

47. A system according to claim 46, wherein the first and second offset values are determined from an observed respiratory rhythm.

48. A system according to claim 46, wherein the airway opening stimulation includes stimulation of the genioglossus muscle.

49. A system according to claim 46, wherein the airway opening stimulation includes stimulation of the hypoglossal nerve.

50. A system according to claim 46, wherein the first electrode includes a cuff electrode assembly adapted to surround part of the vagus nerve of the subject.

51. A system according to claim 50, wherein the cuff electrode assembly is provided with multiple chambers having electrodes therein.

52. A system according to claim 46, further comprising an external control unit including a transceiver for communicating with the transceiver of the implantable control unit, the external control unit allowing interaction with the implantable control unit.

53. A system according to claim 52, wherein the external and implantable control units further include respective power interfaces for transferring power from the external control unit to the implantable control unit.

54. A system according to claim 46, wherein the implantable control unit further includes a power source.

55. A system for detecting hypopnea during the respiratory activity of a subject, comprising:
an electrode for recording an electroneurogram signal from the vagus nerve of the subject;
a transceiver;
an implantable control unit operatively connected to the electrode and the transceiver, the implantable control unit including:
a signal amplifier for amplifying the electroneurogram signal;
a rectifier for rectifying the amplified electroneurogram signal;
a monitoring and detection module for:
computing an amplitude envelope of the amplified electroneurogram signal;
applying a matched filter to the amplitude envelope;
computing a deviation of the signal resulting from the application of the matched filter to the amplitude envelope from a predictor, the predictor being based on previously obtained respiration activity;
transmitting the occurrence of a hypopnea event using the transceiver should the deviation be greater than a hypopnea event threshold.

56. A system according to claim 55, wherein the deviation is an increase in amplitude.

57. A system according to claim 55, wherein the deviation is an increase in respiratory rhythm.

58. A system according to claim 55, wherein the electrode includes a cuff electrode assembly adapted to surround part of the vagus nerve of the subject.

59. A system according to claim 58, wherein the cuff electrode assembly is provided with multiple chambers having electrodes therein.

60. A system according to claim 55, further comprising an external control unit including a transceiver for communicating with the transceiver of the implantable control unit, the external control unit allowing interaction with the implantable control unit.

61. A system according to claim 60, wherein the external and implantable control units further include respective power interfaces for transferring power from the external control unit to the implantable control unit.

62. A system according to claim 55, wherein the implantable control unit further includes a power source.

63. A method for monitoring the respiratory activity of a subject, comprising the steps of:
recording an electroneurogram signal from the vagus nerve of the subject;
amplifying the electroneurogram signal;
extracting respiratory activity information from the amplified signal; and
providing the extracted respiratory activity information.

64. A system for monitoring the respiratory activity of a subject, comprising:
an electrode for detecting an electroneurogram signal from the vagus nerve of the subject;
a transceiver;
an implantable control unit operatively connected to to the electrode and the transceiver, the implantable control unit including:
a signal amplifier for amplifying the electroneurogram signal;
a rectifier for rectifying the amplified electroneurogram signal;
a monitoring and detection module for:
extracting respiratory activity information from the amplified signal; and
transmitting the extracted respiratory activity information using the transceiver.
